(19) United States
(12) Patent Application Publication
(10) Pub. No.: US 2021/0282949 A1
(43) Pub. Date: Sep. 16, 2021

(54) MEDICAL INSTRUMENT FOR INTRALUMINAL INSERTIONS

(71) Applicants: Felice Pecoraro, Palermo (IT); Massimiliano Zingales, Patti (IT); Emanuela Bologna, Palermo (IT)

(72) Inventors: Felice Pecoraro, Palermo (IT); Massimiliano Zingales, Patti (IT); Emanuela Bologna, Palermo (IT); Guido Bajardi, Palermo (IT)

(21) Appl. No.: 17/255,565

(22) PCT Filed: Jun. 26, 2019
(86) PCT No.: PCT/IB2019/055408

§ 371 Date: Dec. 23, 2020

(30) Foreign Application Priority Data
Jun. 29, 2018 (IT) 102018000006821

(51) Int. Cl. A61F 2/954; A61F 2/07

(52) U.S. Cl. CPC A61F 2/954; A61F 2002/061; A61F 2/07

(57) ABSTRACT

A medical instrument for intraluminal insertions, comprising:

a main tubular cannulation body (9) with a tubular wall provided with one or more side openings (13-16);

a main catheter (7) crossed by a central channel (6) and intended to be inserted in the main channel of the lumen (18) to be cannulated;

one or more secondary catheters (2-5) passing through respective side openings (13-16) of said tubular wall;

means for inserting and guiding said main tubular body (9) into the lumen (18) to be cannulated, with a release device of the main tubular body (9) provided with a conical tip (1) distally associated with said main catheter (7) and provided with an axial passage coaxial with said central channel (6) in order to facilitate the insertion of said release device into the lumen (18) and a tubular containment casing (8) peripherally placed and coaxially to said main tubular body (9) in order to define an annular chamber (30) adapted to receive at least partially said one or more secondary catheters (2-5).

### DESCRIPTION

### Field of Application

The present invention finds application in the field of medical devices that can be inserted inside the human body and has particularly as object a medical instrument for intraluminal insertions, such as devices for cannulation or catheterization of the vascular system.

### State of the Art

The use of instruments such as stents and grafts is known for the endovascular treatment of pathologies affecting lumen organs and in particular the vascular system, such as the aneurysmal disease, in which the endovascular approach has proved to be more efficient than traditional surgical approaches.

Typically, these instruments consist of an internal metal skeleton surrounded by an outer plastic membrane and are configured to expand once inserted inside the blood vessel in order to maintain its dilation.

Their insertion is usually performed with the help of guide wires inserted inside the lumen through a common access, chosen according to the type of operation to be performed. For example, US patent application published with nr. US2015173923 describes a system comprising: a release catheter having a flexible catheter body, a proximal end and a distal end; an endoluminal prosthesis comprising a graft of a main body having a lumen and a plurality of fenestrations in a wall portion of the main graft body. The fenestrations are offset from one another along a length of the graft of the main body. Also present are a plurality of sheaths positioned through at least a portion of the lumen of the main graft body and through a corresponding window in the wall portion of the main graft body when the endoluminal prosthesis is in a collapsed pre-dispensing condition within the catheter body.

One of the most common difficulties in the use of such known instruments is found in the cases in which need to proceed with the cannulation of collateral branches, as in the case of thoracic abdominal or aortic arch aneurysms.

In fact, in these situations the standard type stent graft does not allow to maintain blood circulation even in collateral vessels.

To this end, fenestrated or branched stent grafts are used, the fenestrations or branches of which are brought in correspondence with the origin of the collateral vessels in such a way to be able to cannulate the latter, if necessary, by means of a bridged stent graft.

However, the implementation of the cannulation of the collateral vessels after the insertion of the main stent graft has proved to be particularly complex and difficult.

### Presentation of the Invention

The object of the present invention is to overcome the drawbacks indicated above by providing a medical instrument for intraluminal insertions, in particular of the stent-graft type, which is characterized by high efficiency and relative inexpensiveness.

A particular purpose is to provide a medical instrument for intraluminal insertions which allows catheters or cannulas to be inserted into lateral vasculature in a simplified manner in accordance with traditional techniques.

These objects, as well as others which will become clearer below, are achieved by a medical instrument for intraluminal insertions which, according to claim 1, comprises a main tubular cannulation body having a tubular wall provided with one or more side openings, a main catheter crossed by a central channel for being inserted in the main channel of the lumen to be cannulated, one or more secondary catheters passing through respective side openings of said tubular wall, means for inserting and guiding said main tubular body into the lumen to be cannulated.

The insertion and guide means comprise a device for releasing said main tubular body, said release device having a conical tip distally associated with said main catheter and provided with an axial passage coaxial with said central channel, in order to facilitate the insertion of said release device inside the lumen and a tubular containment enclosure peripherally arranged and coaxial with said main tubular body in order to define an annular chamber adapted to accommodate at least partially said one or more secondary catheters.

Thanks to this particular combination of characteristics it will be possible to perform the catheterization of the fenestrations or of the side branches before the insertion of the main stent-graft.

In the present text the terms “distal” and “proximal” are to be interpreted in relation to the position of the parts to which they refer with respect to the operator acting on the device, therefore the term “distal” will indicate the parts being furthest from the hands of the operator, whereas the term “proximal” will indicate the nearest parts.

Advantageous embodiments of the invention are obtained according to dependent claims.

### BRIEF DESCRIPTION OF THE DRAWINGS

Further characteristics and advantages of the invention will become more evident from the detailed description of a preferred but not exclusive embodiment of the instrument according to the invention, illustrated by way of a non-limitative example, with the aid of the combined drawing tables in which:

FIG. 1 is a sectioned side view of the instrument according to the invention;

FIG. 2 is a front view of a detail of the instrument of FIG. 1;

FIG. 3 is a cross-section of the instrument of FIG. 1 according to the tracing plane 3-3;

FIG. 4 is a cross-section of the instrument of FIG. 1 according to the tracing plane 4-4;

FIG. 5 is a cross-section of the instrument of FIG. 1 according to the tracing plane 5-5;

FIGS. from 6 to 9 represent the instrument according to the invention in four distinct operative phases.

### DETAILED DESCRIPTION OF A PREFERRED EMBODIMENT

With reference to the attached figures, a preferred but not exclusive configuration of a medical instrument for intraluminal insertions according to the invention is shown, which is intended to be used in combination with one or more guide wires adapted to be inserted in a lumen of the human body and preferably with lumina having side branches.

In particular, the instrument according to the invention will be used for catheterization operations of blood vessels, although it may be equally used in different organs, possibly without proceeding to the cannulation of secondary or side lumina in the case in which this should not be necessary.

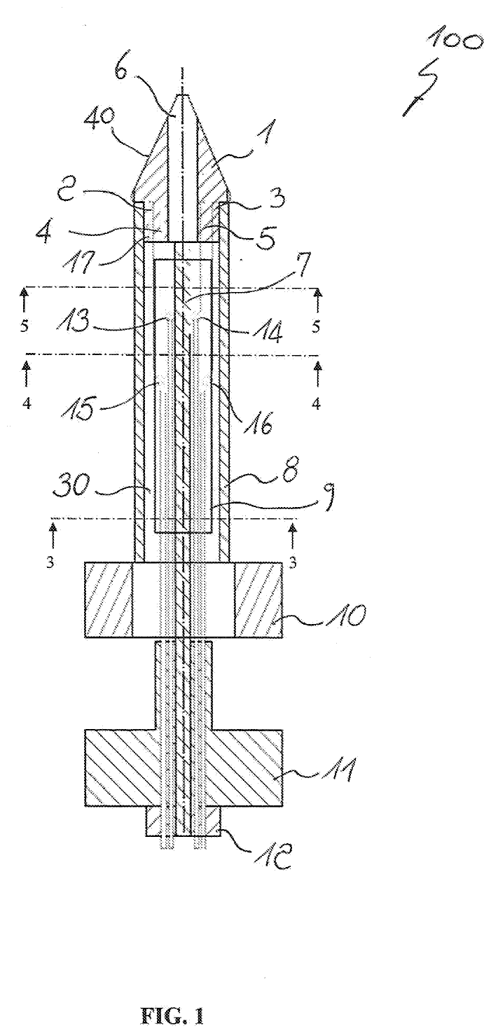

From FIG. 1 it is observed that the instrument, generally indicated by 100, comprises a main tubular cannulation body 9 having a tubular wall provided with one or more lateral openings (four openings 13, 14, 15, 16), and wherein a main catheter 7 is inserted, which is crossed by a central channel 6 which is intended to be inserted in the main channel of the lumen to be cannulated, in particular in a blood vessel 18 such as the aorta, and which will allow the passage of the main guide wire.

Further the device comprises one or more secondary catheters (four catheters 2, 3, 4, 5 in the illustrated configuration), passing through respective side openings 13-16 of the tubular wall of the main body 9.

It is understood that the number of secondary catheters may vary according to the requirements without particular limitations from a theoretical point of view.

In the illustrated exemplary but non-limiting configuration of the invention, the side openings 13-16 are fenestrations made in the side wall of the tubular main body 9.

According to some variants, the fenestrations 13-16 may be single, double or triple for the passage of respective secondary single, double or triple thin catheters 2-5.

Still according to a further variant, instead of the fenestrations 13-16 the main tubular body 9 may present short branches or sides protruding laterally and externally from its lateral wall and oriented in the distal direction. In this configuration the secondary catheters 2-5 will be positioned inside the short branches in order to enter inside the main tubular body 9.

In general, means are also provided for inserting and guiding the main tubular body 9 inside the lumen 18 to be cannulated.

Such insertion and guide means comprise a release device of the main tubular body 9 provided with a conical tip 1 associated with the distal end of the main catheter 7, preferably integral with the same, and provided with a axial passage coaxial with the central channel 6 in order to facilitate the insertion of the release device inside the lumen 18.

The release device is also provided with a tubular containment casing 8 arranged peripherally and coaxial with the main tubular body 9 in order to define an annular chamber 30 adapted to at least partially receive the secondary catheters 2-5.

Advantageously, the main tubular body 9 is an elastically expandable stent-graft arranged inside the tubular containment casing 8 in the initial position which, when it is not released, is radially compressed.

The conical tip 1 is made of a distal portion 40 of conical shape in continuity with a proximal portion 17 of cylindrical shape.

The base of the cylindrical proximal portion 17, which is not in continuity with the distal conical portion 40, may have a blunt conformation and be in continuity with the central catheter 7.

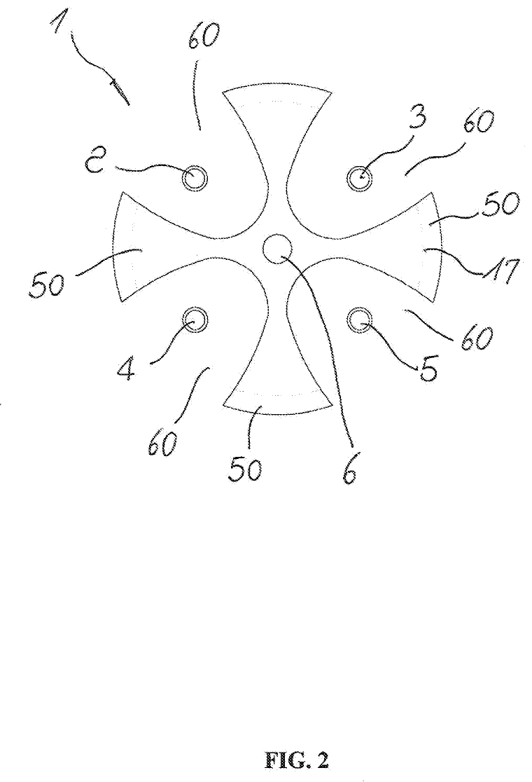

The base of the conical portion 40 is wider than the cylindrical portion 17 and the difference in diameter is equivalent to the thickness of the tubular casing 8, so that the latter does not protrude radially with respect to the tip 1, so facilitating its insertion. As shown in FIG. 2, the tip 1 is cross-shaped in a plan view with four terminals 50, in which each of them widens towards the peripheral edge of the conical portion 40 and with a proximate edge of a semicircular shape. In this way four axial lateral channels 60 are formed in the tip 1 for the passage of respective secondary catheters 2-5, which will thus fit inside the tubular casing 8.

When the main stent-graft 9 is not released, i.e. it is maintained within the tubular casing 8 as in FIG. 1, the tip 1 will have its cylindrical portion 17 positioned within the outer casing 8.

The channels 60 between each terminal 50 of the tip 1 allow the secondary catheters 2-5 to lie at this level between the tubular casing 8 and the tip 1.

Each channel 60 formed between two terminals 50 of the tip 1 allows the passage of a single secondary catheter 2-5.

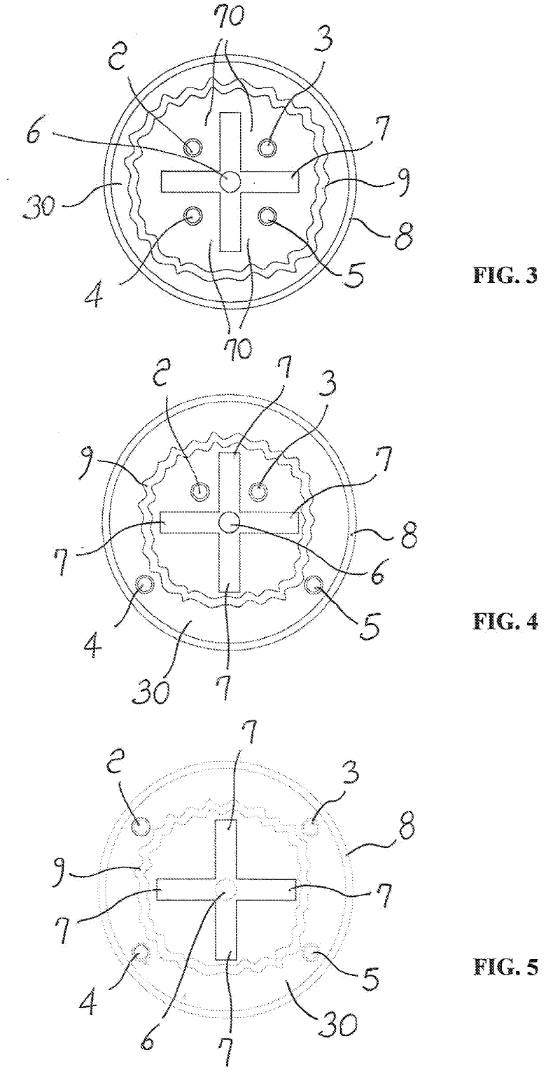

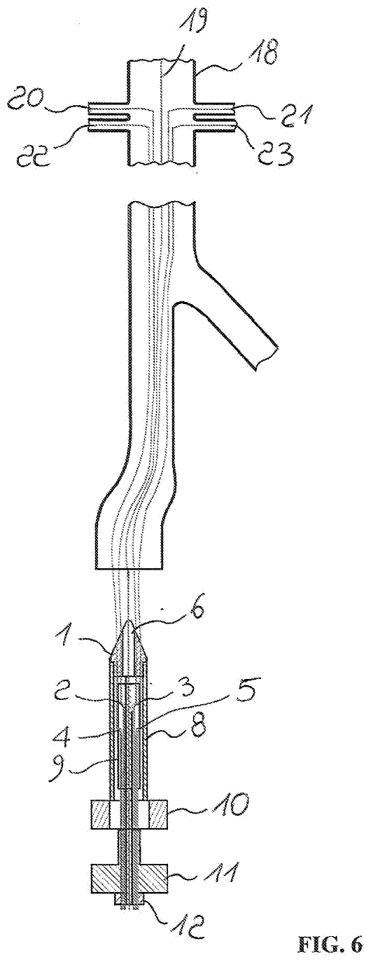

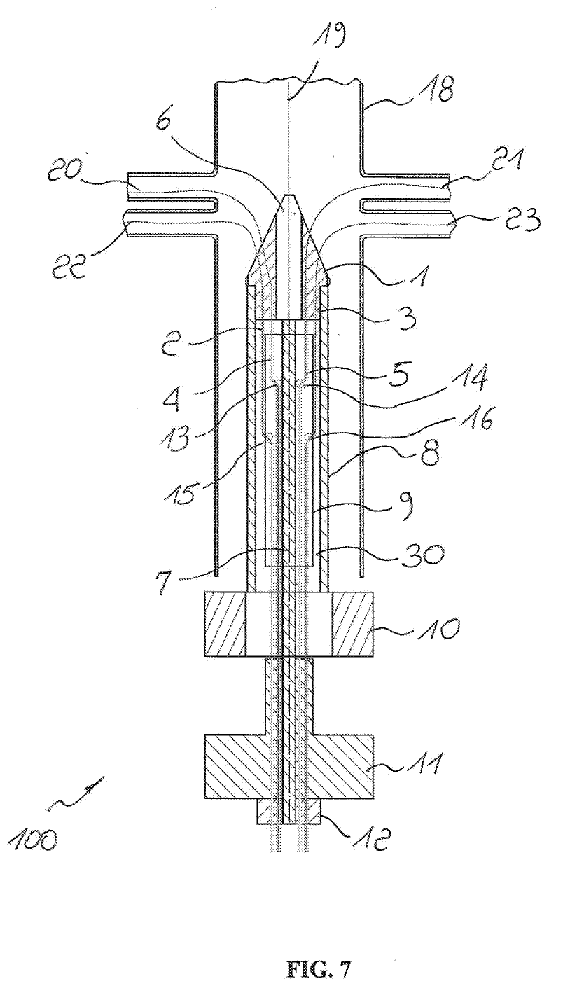

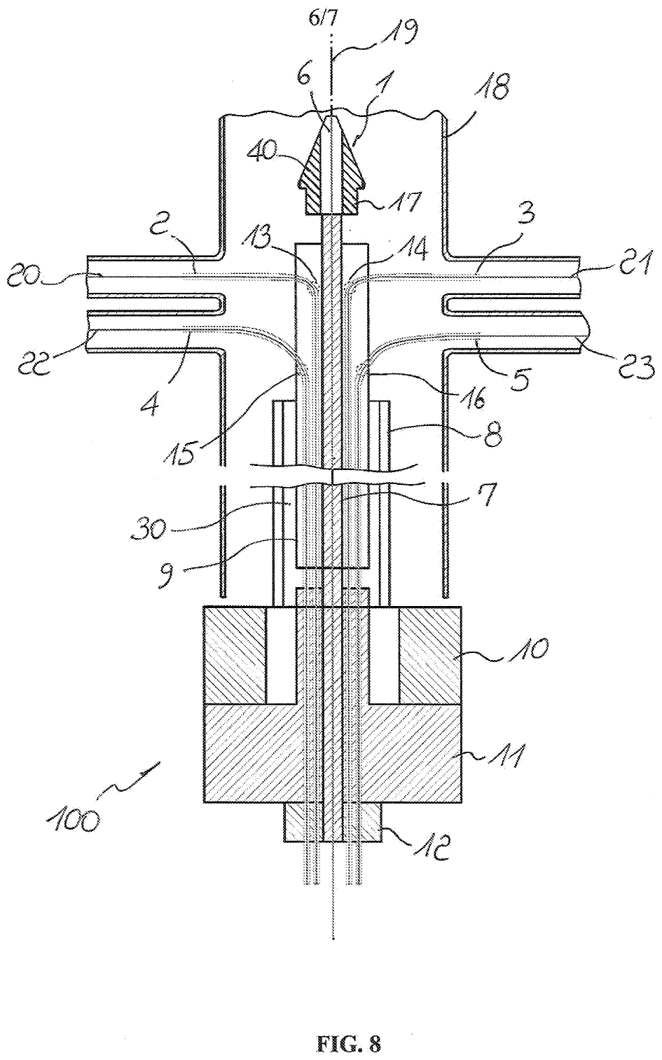

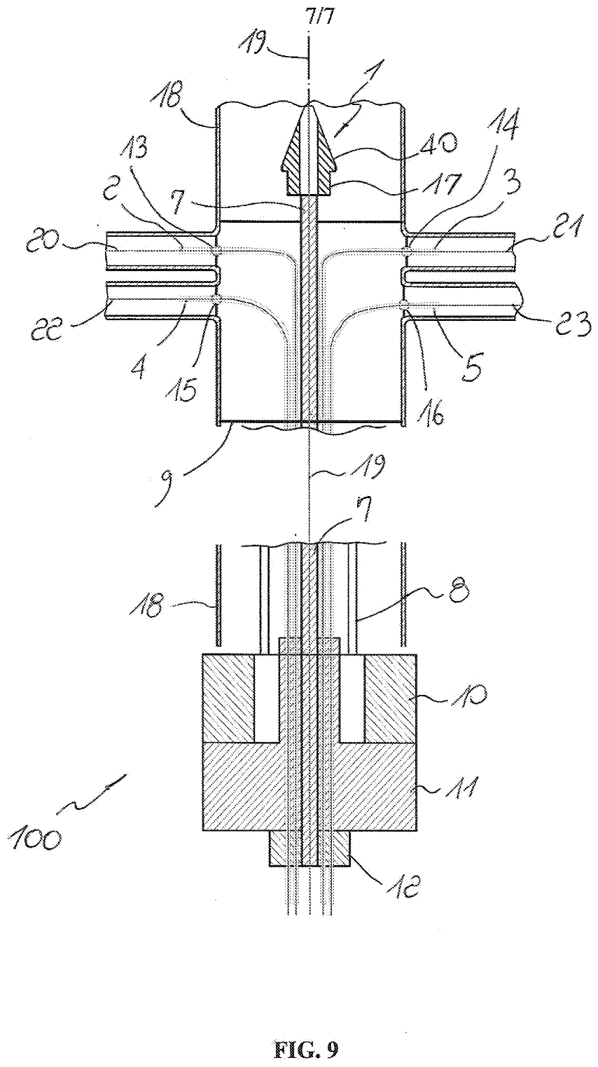

As can be seen from the cross-sections of FIGS. 3-5, each of the secondary catheters 2-5 comprises a distal portion arranged within the annular chamber 30 and outside the main stent-graft 9, an intermediate section crossing a respective fenestration 13-16 of the main stent-graft 9 and a proximal section sliding inside the main stent-graft 9, coaxially therewith and externally to the main catheter 7.

In particular, from FIG. 4 it can be observed that in the intermediate cross-section formed between two axially staggered pairs of fenestrations 13-16, two secondary catheters 2, 3 will be present, sliding inside the main stent-graft 9 and two secondary catheters 4, 5 sliding inside the annular chamber 30.

From the same figures it can also be observed that the main catheter 7 has a cross-shaped cross-section in order to define a plurality of lateral channels 70 for the passage of respective distal sections of the secondary catheters 2-5.

The release device also comprises an advancement knob 11 integral with or in any case associated with the main catheter 7 at the end proximal of the same for its axial movement.

The advancement knob 11 is also connected to a release knob 12, arranged proximally to the advancement knob 11 and associated with the main stent-graft 9 in order to allow its release from the tubular containment casing 8 once the instrument has been inserted into the selected lumen.

Preferably, the advancement knob 11 and the release knob 12 are integral with each other in their respective axial movement and comprise respective axial passages for the main catheter 7 and one or more lateral passages for respective secondary catheters 2-5.

The latter protrude proximally about 10-20 centimeters from the release knob 12 and one or more of them can be removed from the proximal portion of the release device.

The release device also comprises a gripping element or knob 10 distally arranged with respect to the previous knobs 11, 12 and integral with the tubular containment casing 8 in order to be able to keep it steady during the axial advancement of the main catheter 7 facilitated by the advancement knob 11.

In FIGS. 6 to 9 a sequence is shown of the insertion of the instrument 100 inside a lumen of the human body and in particular inside a blood vessel 18 provided with lateral branches, such as the aorta and visceral arteries.

In this exemplary case, the main guide 19 is positioned at the ascending aorta and the four collateral guides 20, 21, 22, 23 are positioned in the corresponding visceral arteries with a traditional method.

The proximal portion of the main guide 19 and of the collateral guides 20-23 are inserted in a retrograde manner respectively in the central channel 6 and in the side catheters 2-5 outside the human body from a single access, for example a femoral or iliac one.

The main guide 19 and the collateral guides 20-23 are recaptured outside the proximal portion of the secondary catheters 2-5.

In FIG. 7 the insertion is shown inside the human body of the main stent-graft 9 which is not released.

The advancement of the main stent-graft 9 inside the human body is realized with the main guide 19 which is introduced inside the central channel 6.

At the same time the collateral guides 20-23 are arranged inside the secondary catheters 2-5. At this moment the grip knob 10, the advancement knob 11 and the release knob 12 are in the initial position.

During these advancing movements, the main guide 19 and the collateral guides 20-23 are maintained respectively in the ascending aorta and in the collateral vessels by applying a firm tension at the proximal ends outside the human body.

This advancing movement is interrupted when the base of the conical part of the tip 1 is positioned 1-2 centimeters below the lower, that is collateral, proximal vessel.

In FIG. 8 the advancement is shown of the stent-graft 9 which is not released upon the main guide 19, which is done by keeping the outer casing 8 below the lower collateral vessel. This movement is carried out by axially advancing the advancement knob 11 and the release knob 12 and keeping the grip knob 10 firm.

This movement ends when the fenestrations 13-16, or the branches or sides, are positioned in correspondence with the respective collateral vessels.

With this movement, the outer casing 8, in continuity with the grip knob 10, is kept about 1-2 centimeters below the lower collateral vessel.

The same movement of the advancement knob 11 advances the tip 1, the central catheter 7 and the stent-graft 9 and also determines the release of the cylindrical portion 17 of the tip 1 with respect to the outer casing 8.

This movement also determines the release of the collateral guides 20-23 in the annular chamber 30 between the outer casing 8 and the stent-graft 9 up to the corresponding fenestrations 13-16.

During this movement the secondary catheters 2-5 can be kept in position or proximally or distally moved above the respective collateral guide 20-23 according to the specific anatomy and needs.

As an alternative, the secondary catheters 2-5 can be removed from the proximal portion of the instrument before the stent-graft 9 advances on the main guide 19, still maintaining the outer casing 8 below the lower collateral vessel.

FIG. 9 shows the stent-graft 9 released with the use of the release knob 12, carried out while maintaining the side guides 20-23 inside the collateral vessels.

The secondary catheters 2-5 can be placed inside the collateral vessels or be removed during release of the stent-graft 9.

The instrument according to the invention is susceptible of numerous modifications and variants, all within the inventive concept set out in the appended claims. All the details may be replaced by other technically equivalent elements, and the materials may be different according to requirements, without departing from the scope of the present invention.

Although the instrument has been described with particular reference to the attached figures, the reference numbers used in the description and in the claims are made in order to improve the comprehension of the invention and do not limit in any case the scope of protection claimed.

1. A medical instrument for intraluminal insertions, used in combination with one or more guide wires able to be inserted in a lumen of the human body, such as a blood vessel, said instrument comprising:
a main tubular cannulation body with a tubular wall provided with one or more side openings;
a main catheter crossed by a central channel and intended to be inserted in the main channel of the lumen to be cannulated;
one or more secondary catheters passing through respective side openings of said tubular wall;
means for inserting and guiding said main tubular body into the lumen to be cannulated;
wherein said insertion and guide means comprise a device for releasing said main tubular body, said release device having a conical tip distally associated with said main catheter and provided with an axial passage coaxial with said central channel in order to facilitate the insertion of said release device into the lumen and a tubular containment casing peripherally placed and coaxially to said main tubular body in order to define an annular chamber adapted to receive at least partially said one or more secondary catheters,
characterized in that said release device comprises:
an advancement knob associated with said main catheter for its axial movement, and
a release knob associated with said main tubular body in order to promote its release from said tubular containment casing.

2. The instrument according to claim 1, characterized in that said main tubular body is an elastically expandable stent-graft placed in said tubular containment casing in a radially compressed initial position.

3. The instrument according to claim 1, characterized in that said conical tip has one or more axial side channels for the passage of respective secondary catheters inside said tubular containment casing.

4. The instrument according to claim 1, characterized in that each of said secondary catheters comprises a distal portion placed in said annular chamber outside said central tubular body, an intermediate portion which crosses a respective one of said one or more side openings of said main tubular body and a proximal portion which slides inside said main tubular body, coaxially with said central channel.

5. The instrument according to claim 4, characterized in that said main catheter has a cross-shaped section in order to define a plurality of side channels for the passage of respective distal portions of said one or more secondary catheters.

6. The instrument according to claim 1, characterized in that said one or more side openings are fenestrations made in said side wall of said tubular main body.

7. The instrument according to claim 1, characterized in that said one or more side openings are associated with branches laterally and externally protruding from said side wall of said tubular main body.

8. The instrument according to claim 1, characterized in that said advancement knob and said release knob are integral with each other in the respective axial movement and comprise respective axial passages for said main catheter and one or more side passages for respective of said secondary catheters.

9. The instrument according to claim 8, characterized in that said release device comprises a grip element distally placed with respect to said knobs and integral with said tubular containment casing in order to allow to maintain said tubular containment casing firm during the axial advancement of said main catheter.

10. The instrument according to claim 2, characterized in that said conical tip has one or more axial side channels for the passage of respective secondary catheters inside said tubular containment casing.

11. The instrument according to claim 2, characterized in that each of said secondary catheters comprises a distal portion placed in said annular chamber outside said central tubular body, an intermediate portion which crosses a respective one of said one or more side openings of said main tubular body and a proximal portion which slides inside said main tubular body, coaxially with said central channel.

12. The instrument according to claim 3, characterized in that each of said secondary catheters comprises a distal portion placed in said annular chamber outside said central tubular body, an intermediate portion which crosses a respective one of said one or more side openings of said main tubular body and a proximal portion which slides inside said main tubular body, coaxially with said central channel.

13. The instrument according to claim 2, characterized in that said one or more side openings are fenestrations made in said side wall of said tubular main body.

14. The instrument according to claim 3, characterized in that said one or more side openings are fenestrations made in said side wall of said tubular main body.

15. The instrument according to claim 4, characterized in that said one or more side openings are fenestrations made in said side wall of said tubular main body.

16. The instrument according to claim 5, characterized in that said one or more side openings are fenestrations made in said side wall of said tubular main body.

17. The instrument according to claim 2, characterized in that said one or more side openings are associated with branches laterally and externally protruding from said side wall of said tubular main body.

18. The instrument according to claim 3, characterized in that said one or more side openings are associated with branches laterally and externally protruding from said side wall of said tubular main body.

19. The instrument according to claim 4, characterized in that said one or more side openings are associated with branches laterally and externally protruding from said side wall of said tubular main body.

20. The instrument according to claim 5, characterized in that said one or more side openings are associated with branches laterally and externally protruding from said side wall of said tubular main body.
